(19) United States
(12) Patent Application Publication
(10) Pub. No.: US 2012/0247498 A1
(43) Pub. Date: Oct. 4, 2012

(54) COSMETIC COMPOSITION COMPRISING A SURFACTANT, A LIQUID FATTY ALCOHOL AND A NONIONIC ASSOCIATIVE POLYMER, AND COSMETIC TREATMENT METHOD

(75) Inventor: Estelle Mathonneau, Paris (FR)

(73) Assignee: L'OREAL, Paris (FR)

(21) Appl. No.: 13/515,886

(22) PCT Filed: Dec. 14, 2010
(86) PCT No.: PCT/FR2010/052705

§ 371 Date: Jun. 14, 2012

(30) Foreign Application Priority Data
Dec. 17, 2009 (FR) 0959124
Dec. 23, 2009 (US) 61289632

(51) Int. Cl. A61K 8/72; A61Q 5/02; A61K 8/89; A61K 8/73; A61K 8/90; A61K 8/87; A61Q 5/12

(52) U.S. Cl. 132/202; 424/70.11; 510/119; 510/127; 510/121; 424/70.12

(57) ABSTRACT

The present invention relates to a composition for washing and conditioning keratin materials, comprising, in a physiologically acceptable medium:
from 3% to 50% by weight, relative to the total weight of the composition, of one or more anionic surfactants,
one or more C₁₀-C₃₀ liquid alcohols chosen from branched and/or unsaturated C₁₀-C₃₀ alcohols, and
one or more nonionic associative polymers.

The invention also relates to the use of the said composition for washing and conditioning keratin materials, and to a process for washing and conditioning keratin materials using it.

The present invention relates to a composition for washing and conditioning keratin materials, in particular keratin fibres, comprising an anionic surfactant, a liquid fatty alcohol and a nonionic fixing polymer, to the use of the said composition for washing and conditioning keratin materials, in particular keratin fibres, and also to a process using the said composition.

In the field of conditioning shampoos, a washing base is generally combined with a conditioning agent, which may be a cationic polymer, an amphoteric polymer, a silicone, a synthetic or natural oil, a fatty substance or a mixture thereof. These conditioning agents are used for improving the disentangling and softness of wet and dried hair, but may have a tendency to make the hair lank and dull.

The use of insoluble conditioning agents is greatly limited:
firstly due to the stabilization difficulties in detergent compositions, if it is desired to maintain the level of the working qualities (stability, latherability, start of lathering),
secondly due to cosmetic defects in terms of the lank, charging and regreasing effect associated with coarse or heterogeneous dispersions in these same detergent compositions.

Insoluble conditioning agents, in particular liquid fatty alcohols, are known and used in shampoo compositions, especially in documents JP2002-20791, JP9-30938, US 2009/005 449 and US 2009/005 460.

However, these compositions are not thickened and/or stable and/or do not have high-quality cosmetic performance in terms of disentangling and smoothing, without making the head of hair lank or making it feel charged, and while maintaining the level of the working qualities.

Specifically, thickened products that can be metered out and taken up easily in the hand are sought. To do this, these products must have a certain consistency or viscosity. Specifically, a liquid product is much more difficult to meter out and flows easily between the fingers.

Natural polymers such as celluloses have already been used to thicken washing compositions. Unfortunately, these thickeners give unstable compositions and/or compositions that are not smooth and homogeneous. Furthermore, these thickeners have the drawback of reducing the quality of the lather and the cosmetic performance of shampoos, especially by making the hair feel more charged and coarser. The lather of thickened compositions is generally not sufficiently soft, and it does not develop easily, either in terms of speed or abundance.

Thus, there is still need for a thickening system that can suitably thicken a washing composition comprising a liquid fatty alcohol without having an impact on the cosmetic and lathering properties of the said compositions, or else while improving them.

The Applicant has now discovered that the use of a nonionic associative polymer in compositions comprising an anionic surfactant and a liquid fatty alcohol makes it possible to overcome the drawbacks indicated above, and thus to obtain a stable composition of homogeneous and aesthetic aspect. Furthermore, the compositions show a good abundance of lather. Moreover, the cosmetic properties and in particular the smoothness, softness, suppleness and sheen are improved. In the case of frizzy and/or curly hair, a decrease in volume is observed, enabling better control of the head of hair.

The compositions according to the invention also afford colour protection on washing artificially dyed hair.

A subject of the invention is thus a composition, especially for washing and conditioning keratin materials, in particular keratin fibres, comprising, in a cosmetically acceptable medium:
from 3% to 50% by weight, relative to the total weight of the composition, of one or more anionic surfactants,
one or more C₁₀-C₃₀ liquid alcohols chosen from branched and/or unsaturated C₁₀-C₃₀ alcohols, and
one or more nonionic associative polymers.

Another subject of the invention is the use of the said composition for washing and conditioning keratin materials, in particular keratin fibres and more particularly the hair.

A subject of the invention is also a process for washing and conditioning keratin fibres using the composition according to the invention.

Other subjects, characteristics, aspects and advantages of the invention will emerge even more clearly on reading the description and the various examples that follow.

In the present patent application, a species is termed as being “anionic” when it bears at least one permanent negative charge or when it can be ionized as a negatively charged species, under the conditions of use of the compositions of the invention (for example the medium or the pH) and not comprising any cationic filler.

A species is termed as being “nonionic” when it is neither cationic nor anionic within the meaning of the present patent application, in particular when it comprises no cationic or anionic groups within the meaning of the present patent application.

The terms “cosmetically acceptable” and “physiologically acceptable” mean compatibility with application to the body of a living being, in particular the human body, especially to the scalp and the hair.

The anionic surfactants that may be used in the composition are especially chosen from salts, in particular the alkali metal salts such as the sodium salts, the ammonium salts, the amine salts, the amino alcohol salts or the salts of alkaline-earth metals, for example of magnesium, of compounds of the following types: alkyl sulfates, alkyl ether sulfates, alkylamido ether sulfates, alkylaryl polyether sulfates, monoglyceride sulfates; alkylsulfonates, alkylamidesulfonates, alkylarylsulfonates, α-olefin sulfonates, paraffin sulfonates; alkyl sulfosuccinates, alkyl ether sulfosuccinates, alkylamide sulfosuccinates; alkyl sulfoacetates; acylsarcosinates; and acylglutamates, the alkyl and acyl groups of all these compounds comprising from 6 to 24 carbon atoms and the aryl group preferably denoting a phenyl or benzyl group.

It is also possible to use monoesters of C₆₋₂₄ alkyl and of polyglycoside-polycarboxylic acids, such as alkyl glucoside citrates, alkyl polyglycoside tartrates and alkyl polyglycoside sulfosuccinates, alkyl sulfosuccinamates, acylisethionates and N-acyltaurates, the alkyl or acyl group of all these compounds comprising from 12 to 20 carbon atoms.

Another group of anionic surfactants that may be used in the composition of the present invention is that of acyl lactylates, the acyl group of which comprises from 8 to 20 carbon atoms.

In addition, mention may also be made of alkyl-D-galactosideuronic acids and salts thereof and also polyoxyalkylenated (C₆₋₂₄)alkyl ether carboxylic acids, polyoxyalkylenated (C₆₋₂₄)alkyl(C₆₋₂₄)aryl ether carboxylic acids, polyoxyalkylenated (C₆₋₂₄)alkylamido ether carboxylic acids and salts thereof, in particular those comprising from 2 to 50 ethylene oxide units, and mixtures thereof.

Preference is given to using alkyl sulfates, alkyl ether sulfates and alkyl ether carboxylates, and mixtures thereof, especially in the form of salts with alkali metals or alkaline-earth metals, with ammonium, with amine or with amino alcohol.

The anionic surfactant(s) are present in a total amount ranging from 3% to 50% by weight and better still from 4% to 20% by weight relative to the total weight of the composition.

The “liquid fatty alcohols” are liquid at room temperature (25° C.) and at atmospheric pressure (1 atm) and are insoluble in water (i.e. they have a solubility in water of less than 1% by weight and preferably less than 0.5% by weight) and are soluble, under the same temperature and pressure conditions, in at least one organic solvent (for example ethanol, chloroform, benzene or liquid petroleum jelly) to at least 1% by weight.

The liquid fatty alcohols, in particular the C₁₀-C₃₀ alcohols, have branched carbon-based chains or contain one or more (preferably 1 to 3) unsaturations.

The fatty alcohols according to the invention are preferably branched and/or unsaturated, and comprise from 12 to 40 carbon atoms. The fatty alcohols of the invention are non-oxyalkylenated and non-glycerolated.

The liquid fatty alcohols preferably have the structure R—OH, in which R preferably denotes a C₁₂-C₂₄ branched alkyl or C₁₂-C₂₄ alkenyl group. R may be substituted with one or more hydroxyl groups. Preferably, R does not contain any hydroxyl groups.

Examples that may be mentioned include oleyl alcohol, linoleyl alcohol, linolenyl alcohol, isocetyl alcohol, isostearyl alcohol, 2-octyl-1-dodecanol, 2-butyloctanol, 2-hexyl-1-decanol, 2-decyl-1-tetradecanol and 2-tetradecyl-1-cetanol, and mixtures thereof.

Preferably, the liquid fatty alcohol of the invention is a branched saturated alcohol. Even more preferentially, the liquid fatty alcohol of the invention is 2-octyl-1-dodecanol.

The fatty alcohols may be mixed, which means that several species may coexist in a commercial product, especially of different chain lengths, in the form of a mixture.

The liquid fatty alcohol(s) are generally present in an amount ranging from 0.01% to 10% by weight, preferentially from 0.1% to 5% by weight and better still from 0.5% to 3% by weight, relative to the total weight of the composition.

For the purposes of the present invention, the term “thickened composition” means a composition with a viscosity of at least 25 cps and preferably of at least 50 cps at a temperature of 25° C. and at a shear rate of 1 s⁻¹. These viscosities may be measured with a rheometer with cone-plate geometry.

The composition moreover comprises one or more nonionic associative polymers.

For the purposes of the present invention, the notion of a “polymer” does not include fatty acid esters of polyalkylene glycols or polyoxyalkylenated fatty alcohols.

The term “associative polymers” means polymers whose molecules are capable, in a given medium, preferably an aqueous medium, of reversibly associating with each other or with other molecules. Their chemical structure comprises at least one hydrophilic zone and at least one hydrophobic zone, the latter being characterized by the presence of at least one fatty chain comprising at least 8 carbon atoms and preferably from 8 to 30 carbon atoms.

The polymers in accordance with the invention generally have a weight-average molar mass ranging from 5000 to 10 000 000, more preferentially from 50 000 to 8 000 000 and even more preferentially from 100 000 to 3 000 000.

More particularly, the nonionic associative polymers are chosen from:
(1) celluloses modified with groups comprising at least one fatty chain; of which, examples that may be mentioned include:
   hydroxyethylcelluloses modified with groups comprising at least one fatty chain, such as alkyl, arylalkyl or alkylaryl groups, or mixtures thereof, and in which the alkyl groups are preferably C₈-C₂₂, for instance the product Natrosol Plus Grade 330 CS® (C₁₋₆ alkyl) sold by the company Aqualon, or the product Bermocoll EHM 100® sold by the company Berol Nobel,
(2) hydroxypropyl guars modified with groups comprising at least one fatty chain, such as the product Esaflor HM 22® (C₂₂ alkyl chain) sold by the company Lamberti, and the products RE210-18® (C₁₄ alkyl chain) and RE205-1® (C₂₀ alkyl chain) sold by the company Rhône-Poulenc,
(3) copolymers of vinylpyrrolidone and of fatty-chain hydrophobic monomers,
(4) copolymers of C₁-C₆ alkyl methacrylates or acrylates and of amphiphilic monomers comprising at least one fatty chain, such as, for example, the oxyethylenated methyl acrylate/stearyl acrylate copolymer sold by the company Goldschmidt under the name Antil 208®,
(5) copolymers of hydrophilic methacrylates or acrylates and of hydrophobic monomers comprising at least one fatty chain, such as, for example, the polyethylene glycol methacrylate/lauryl methacrylate copolymer,
(6) polyurethane polyethers comprising in their chain both hydrophilic blocks usually of polyoxyethylenated nature and hydrophobic blocks, which may be aliphatic sequences alone and/or cycloaliphatic and/or aromatic sequences,
(7) polymers with an aminoplast ether backbone containing at least one fatty chain, such as the Pure Thix® compounds sold by the company Sud-Chemie.

Preferably, the polyurethane polyethers comprise at least two hydrocarbon-based lipophilic chains containing from 8 to 30 carbon atoms, separated by a hydrophilic block, the hydrocarbon-based chains possibly being pendent chains or chains at the end of the hydrophilic block.

In particular, it is possible for one or more pendent chains to be included.

In addition, the polymer may comprise a hydrocarbon-based chain at one end or at both ends of a hydrophilic block.

The polyurethane polyethers may be multiblock, in particular in triblock form. The hydrophobic blocks may be at each end of the chain (for example: triblock copolymer containing a hydrophilic central block) or distributed both at the ends and in the chain (for example multiblock copolymer). These same polymers may also be graft polymers or star polymers.

The fatty-chain nonionic polyurethane polyethers may be triblock copolymers in which the hydrophilic block is a polyoxyethylenated chain comprising from 50 to 1000 oxyethylene groups. The nonionic polyurethane polyethers comprise a urethane linkage between the hydrophilic blocks, whence arises the name.

By extension, also included among the fatty-chain nonionic polyurethane polyethers are those in which the hydrophilic blocks are linked to the lipophilic blocks via other chemical bonds.

As examples of fatty-chain nonionic polyurethane polyethers that may be used in the invention, it is also possible to use Rheolate 205® containing a urea function, sold by the company Rheox, or Rheolate® 208, 204 or 212, and also Acrysol RM 184®.

Mention may also be made of the product Elfacos T210® containing a C₁₂-C₁₄ alkyl chain, and the product Elfacos T212® containing a C₁₋₈ alkyl chain, from Akzo.

It is also possible to use solutions or dispersions of these polymers, especially in water or in aqueous-alcoholic medium. Examples of such polymers that may be mentioned are Rheolate® 255, Rheolate® 278 and Rheolate® 244 sold by the company Rheox.

Even more particularly, according to the invention, it is also possible to use a polyurethane polyether that may be obtained by polycondensation of at least three compounds comprising (i) at least one polyethylene glycol comprising from 120 to 180 mol of ethylene oxide, (ii) a C₁₀-C₂₀ fatty alcohol, in particular stearyl alcohol or decyl alcohol, and (iii) at least one diisocyanate.

Such polyurethane polyethers are sold especially by the company Röhm & Haas under the names Aculyn 46® and Aculyn 44® [Aculyn 46® is a polycondensate of polyethylene glycol containing 150 or 180 mol of ethylene oxide, of stearyl alcohol and of methylenebis(4-cyclohexyl isocyanate) (SMDI), at 15% by weight in a matrix of maltodextrin (4%) and water (81%); Aculyn 44® is a polycondensate of polyethylene glycol containing 150 or 180 mol of ethylene oxide, of decyl alcohol and of methylenebis(4-cyclohexyl isocyanate) (SMDI), at 35% by weight in a mixture of propylene glycol (39%) and water (26%)].

According to one particular form of the invention, use will be made of a polyurethane polyether that may be obtained by polycondensation of at least three compounds comprising (i) at least one polyethylene glycol comprising from 100 to 180 mol of ethylene oxide and in particular from 120 to 160 mol of ethylene oxide, (ii) a C16-20 fatty alcohol polyoxyethylenated with 80 to 120 and in particular 90 to 110 mol of ethylene oxide and especially a polyoxyethylenated stearyl alcohol comprising from 80 to 120 and in particular 90 to 110 mol of ethylene oxide, and (iii) an aliphatic diisocyanate, in particular hexamethylene diisocyanate.

Such polyurethane polyethers are sold especially by the company Elementis under the name Rheolate FX 1100®, which is a polycondensate of polyethylene glycol containing about 136 mol of ethylene oxide, of stearyl alcohol polyoxyethylenated with about 100 mol of ethylene oxide and of hexamethylene diisocyanate (HDI) with a weight-average molecular weight of from 25 000 to 35 000 (INCI name: PEG-136/Steareth-100/HDI Copolymer).

Advantageously, the nonionic associative polymer(s) are generally present in an amount ranging from 0.1% to 5% by weight, preferably from 0.5% to 4% by weight, preferentially from 1.5% to 4% by weight and better still from 1.5% to 3% by weight relative to the total weight of the composition.

In one variant of the invention, the weight ratio of nonionic associative polymer(s)/liquid fatty alcohol(s) ranges from 0.005 to 50, preferably from 0.1 to 30, better still from 0.2 to 10 and even more particularly from 0.5 to 5.

The composition may also comprise at least one nonionic surfactant and/or at least one amphoteric surfactant.

The nonionic surfactants which can be used in the compositions of the present invention are compounds well known per se (see in particular in this respect “Handbook of Surfactants” by M. R. Porter, published by Blackie & Son (Glasgow and London), 1991, pp. 116-178). They are especially chosen from polyethoxylated, polypropoxylated or polyglycerolated alcohols, alpha-diols, (C₁₋₂₀)alkylphenols and fatty acids, containing a fatty chain comprising, for example, from 8 to 18 carbon atoms, the number of ethylene oxide or propylene oxide groups possibly ranging especially from 2 to 50, and the number of glycerol groups possibly ranging especially from 2 to 30.

Mention may also be made of condensates of ethylene oxide and of propylene oxide with fatty alcohols; polyethoxylated fatty amides preferably having from 2 to 30 ethylene oxide units, polyglycerolated fatty amides containing on average 1 to 5 glycerol groups, and in particular 1.5 to 4, ethoxylated fatty acid esters of sorbitan containing from 2 to 30 ethylene oxide units, fatty acid esters of sucrose, fatty acid esters of polyethylene glycol, (C₆₋₂₄alkyl)polyglycosides, N—(C₆₋₂₄ alkyl)glucamine derivatives, amine oxides such as (C₁₀₋₁₄alkyl)amine oxides or N—(C₁₀₋₁₄ acyl)aminopropylmorpholine oxides.

Among the nonionic surfactants mentioned above, use is preferably made of (C₆₋₂₄ alkyl)polyglycosides and more particularly (C₈₋₁₈ alkyl)polyglycosides.

The amphoteric surfactants that may be used in the present invention may especially be secondary or tertiary aliphatic amine derivatives in which the aliphatic group is a linear or branched chain containing from 8 to 22 carbon atoms and containing at least one anionic group, for instance a carboxylate, sulfonate, sulfate, phosphate or phosphonate group. Mention may be made in particular of (C₈₋₂₀)alkylbetaines, sulfobetaines, (C₈₋₂₀ alkyl)amido(C₂₋₈ alkyl)betaines and (C₈₋₂₀ alkyl)amido(C₂₋₈ alkyl)sulfobetaines.

Among the amine derivatives that may be mentioned are the products sold under the name Miranol®, as described in patents U.S. Pat. No. 2,528,378 and U.S. Pat. No. 2,781,354 and classified in the CTFA dictionary, 3rd edition, 1982, under the names amphocarboxyglycinate and amphocarboxypropionate, having the respective structures (1) and (2):

Rₐ—CONHCH₂CH₂—N(R_{b})(R_{c})(CH₂COO⁻) (1)

in which:

Rₐ represents an alkyl group derived from an acid Rₐ—COOH present in hydrolysed coconut oil, or a heptyl, nonyl or undecyl group,

R_{b} represents a β-hydroxyethyl group, and

R_{c} represents a carboxymethyl group; and

Rₐ′—CONHCH₂CH₂—N(B)(C) (2)

in which:

B represents —CH₂CH₂OX′,

C represents —(CH₂)_{z}—Y′, with z=1 or 2,

X′ represents the —CH₂CH₂—COOH group or a hydrogen atom,

Y′ represents —COOH or the —CH₂—CHOH—SO₃H group,

Rₐ′ represents an alkyl group of an acid Rₐ′-COOH present in coconut oil or in hydrolysed linseed oil, a saturated or unsaturated C₇-C₂₃ alkyl group, especially a C₁₇ group and its iso form, or an unsaturated C₁₇ group.

These compounds are classified in the CTFA dictionary, 5th edition, 1993, under the names disodium cocoamphodiacetate, disodium lauroamphodiacetate, disodium caprylamphodiacetate, disodium caprylamphodiacetate, disodium cocoamphodipropionate, disodium lauroamphodipropionate, disodium caprylamphodipropionate, disodium caprylamphodipropionate, lauroamphodipropionic acid, cocoamphodipropionic acid.

By way of example, mention may be made of the cocoamphodiacetate sold by the company Rhodia under the trade name Miranol® C2M Concentrate.

Among the amphoteric surfactants mentioned above that are preferably used are (C₈₋₂₀ alkyl)betaines, (C₈₋₂₀ alkyl)amido(C₂₋₈ alkyl)betaines and alkylamphodiacetates, and mixtures thereof.

The nonionic and/or amphoteric surfactant(s) are preferably present in the composition according to the invention in an amount ranging from 0 to 20% by weight and better still from 0.5% to 10% by weight relative to the total weight of the composition.

The total amount of surfactants is preferably between 4% and 50% and better still between 5% and 30% by weight relative to the total weight of the composition.

The composition according to the invention may also especially comprise one or more cationic polymers. The cationic polymers that may be used in accordance with the present invention may be chosen from any of those already known per se as improving the cosmetic properties of hair, namely, especially, those described in patent application EP-A-0 337 354 and in French patent applications FR-A-2 270 846, 2 383 660, 2 598 611, 2 470 596 and 2 519 863.

Even more generally, for the purposes of the present invention, the term “cationic polymer” denotes any polymer comprising cationic groups and/or groups that can be ionized into cationic groups.

The preferred cationic polymers are chosen from those that contain units comprising primary, secondary, tertiary and/or quaternary amine groups that may either form part of the main polymer chain or may be borne by a side substituent directly connected thereto.

The cationic polymers used generally have a number-average or weight-average molar mass of between 500 and 5×10⁶ approximately and preferably between 10³ and 3×10⁶ approximately.

Among the cationic polymers that may be mentioned more particularly are polymers of the polyamine, polyaminoamide and polyquaternary ammonium type. These are known products.

The polymers of polyamine, polyamidoamide and polyquaternary ammonium type, that can be used in accordance with the present invention, and that can in particular be mentioned, are those described in French patents No. 2 505 348 or 2 542 997. Among these polymers, mention may be made of:

(1) homopolymers or copolymers derived from acrylic or methacrylic esters or amides and comprising at least one of the units of the following formulae:

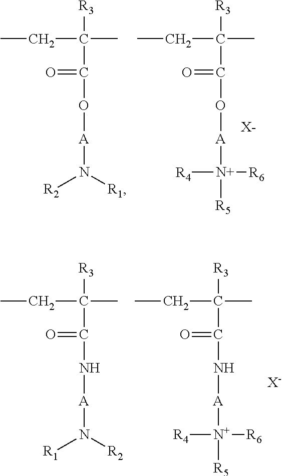

in which:

R₃, which may be identical or different, denotes a hydrogen atom or a CH₃ radical;

A, which may be identical or different, represent a linear or branched alkyl group of 1 to 6 carbon atoms, preferably 2 or 3 carbon atoms, or a hydroxyalkyl group of 1 to 4 carbon atoms;

R₄, R₅ and R₆, which may be identical or different, represent an alkyl group containing from 1 to 18 carbon atoms or a benzyl radical and preferably an alkyl group containing from 1 to 6 carbon atoms;

R₁ and R₂, which may be identical or different, represent hydrogen or an alkyl group containing from 1 to 6 carbon atoms, and preferably methyl or ethyl;

X denotes an anion derived from a mineral or organic acid, such as a methosulfate anion or a halide such as chloride or bromide.

The copolymers of family (1) can also contain one or more units derived from comonomers that may be selected from the family of acrylamides, methacrylamides, diacetone acrylamides, acrylamides and methacrylamides substituted on the nitrogen with lower (C₁-C₄) alkyls, acrylic or methacrylic acids or esters thereof, vinyllactams such as vinylpyrrolidone or vinylcaprolactam, and vinyl esters.

Thus, among these copolymers of the family (1), mention may be made of:
copolymers of acrylamide and of dimethylaminoethyl methacrylate quaternized with dimethyl sulfate or with a dimethyl halide, such as the product sold under the name Hercofloc by the company Hercules,
copolymers of acrylamide and of methacryloyloxyethyltrimethylammonium chloride, described, for example, in Patent Application EP-A-080 976 and sold under the name Bina Quat P 100 by the company Ciba Geigy,

the copolymer of acrylamide and of methacryloyloxyethyltrimethylammonium methosulfate sold under the name Reten by the company Hercules,

quaternized or non-quaternized vinylpyrrolidone/dialkylaminoalkyl acrylate or methacrylate copolymers, such as the products sold under the name Gafquat by the company ISP, such as, for example, Gafquat 734 or Gafquat 755, or alternatively the products known as Copolymer 845, 958 and 937. These polymers are described in detail in French Patents 2 077 143 and 2 393 573,

dimethylaminoethyl methacrylate/vinylcaprolactam/vinylpyrrolidone terpolymers, such as the product sold under the name Gaffix VC 713 by the company ISP,

vinylpyrrolidone/methacrylamidopropyldimethylamine copolymers sold in particular under the name Styleze CC 10 by ISP,

vinylpyrrolidone/quaternized dimethylaminopropylmethacrylamide copolymers such as the product sold under the name Gafquat HS 100 by the company ISP,—preferably crosslinked polymers of methacryloyloxy(C₁-C₄)alkyl tri(C₁-C₄)alkylammonium salts, such as the polymers obtained by homopolymerization of dimethylaminoethyl methacrylate quaternized with methyl chloride, or by copolymerization of acrylamide with dimethylaminoethyl methacrylate quaternized with methyl chloride, the homo- or copolymerization being followed by crosslinking with an olefinically unsaturated compound, more particularly methylenebisacrylamide. A crosslinked acrylamide/methacryloyloxyethyltrimethylammonium chloride copolymer (20/80 by weight) in the form of a dispersion containing 50% by weight of the said copolymer in mineral oil can be used more particularly. This dispersion is sold under the name Salcare® SC 92 by the company Ciba. A crosslinked methacryloyloxyethyltrimethylammonium chloride homopolymer containing about 50% by weight of the homopolymer in mineral oil or in a liquid ester can also be used. These dispersions are sold under the names Salcare® SC 95 and Salcare® SC 96 by the company Ciba.

(2) Cationic polysaccharides, especially cationic celluloses and galactomannan gums. Among the cationic polysaccharides, mention may be made more particularly of cellulose ether derivatives comprising quaternary ammonium groups, cationic cellulose copolymers or cellulose derivatives grafted with a water-soluble quaternary ammonium monomer and cationic galactomannan gums.

The cellulose ether derivatives containing quaternary ammonium groups, described in French patent 1 492 597, and in particular polymers sold under the names Ucare Polymer “JR” (JR 400T, JR 125 and JR 30M) or “LR” (LR 400 or LR 30M) by the company Amerchol. These polymers are also defined in the CTFA dictionary as quaternary ammoniums of hydroxyethyl cellulose that have reacted with an epoxide substituted with a trimethylammonium group.

Cationic cellulose copolymers or cellulose derivatives grafted with a water-soluble monomer of quaternary ammonium are described especially in U.S. Pat. No. 4,131,576, such as hydroxyalkyl celluloses, for instance hydroxymethyl-, hydroxyethyl- or hydroxypropylcelluloses grafted, in particular, with a methacryloylethyltrimethylammonium, methacrylamidopropyltrimethylammonium or dimethyldiallylammonium salt.

The commercial products corresponding to this definition are more particularly the products sold under the names Celquat L 200 and Celquat H 100 by the company National Starch.

The cationic galactomannan gums are described more particularly in U.S. Pat. Nos. 3,589,578 and 4,031,307, in particular guar gums comprising cationic trialkylammonium groups. Guar gums modified with a salt (e.g. chloride) of 2,3-epoxypropyltrimethylammonium are used, for example.

Such products are sold in particular under the trade names Jaguar C13S, Jaguar C 15, Jaguar C 17 or Jaguar C162 by the company Rhodia.

(3) Polymers formed from piperazinyl units and divalent alkylene or hydroxyalkylene radicals containing straight or branched chains, optionally interrupted with oxygen, sulfur or nitrogen atoms or with aromatic or heterocyclic rings, and also the oxidation and/or quaternization products of these polymers. Such polymers are especially described in French Patents 2 162 025 and 2 280 361.

(4) Water-soluble polyaminoamides prepared in particular by polycondensation of an acidic compound with a polyamine; these polyaminoamides can be crosslinked with an epihalohydrin, a diepoxide, a dianhydride, an unsaturated dianhydride, a bisunsaturated derivative, a bis-halohydrin, a bisazetidinium, a bis-haloacyldiamine, a bis-alkyl halide or alternatively with an oligomer resulting from the reaction of a difunctional compound which is reactive with a bis-halohydrin, a bis-azetidinium, a bis-haloacyldiamine, a bis-alkyl halide, an epihalohydrin, a diepoxide or a bis-unsaturated derivative; the crosslinking agent being used in proportions ranging from 0.025 to 0.35 mol per amine group of the polyaminoamide; these polyaminoamides can be alkylated or, if they comprise one or more tertiary amine functions, they can be quaternized. Such polymers are especially described in French Patents 2 252 840 and 2 368 508.

(5) Polyaminoamide derivatives resulting from the condensation of polyalkylene polyamines with polycarboxylic acids followed by alkylation with difunctional agents. Mention may be made, for example, of adipic acid/dialkylaminohydroxyalkyldialkylenetriamine polymers in which the alkyl radical comprises from 1 to 4 carbon atoms and preferably denotes methyl, ethyl or propyl. Such polymers are especially described in French patent 1 583 363.

Among these derivatives, mention may be made more particularly of the adipic acid/dimethylaminohydroxypropyl/diethylenetriamine polymers sold under the name Cartaretine F, F4 or F8 by the company Sandoz.

(6) The polymers obtained by reaction of a polyalkylene polyamine containing two primary amine groups and at least one secondary amine group with a dicarboxylic acid selected from diglycolic acid and saturated aliphatic dicarboxylic acids having from 3 to 8 carbon atoms. The molar ratio between the polyalkylene polyamine and the dicarboxylic acid is between 0.8:1 and 1.4:1; the polyaminoamide resulting therefrom is reacted with epichlorohydrin in a mole ratio of epichlorohydrin relative to the secondary amine group of the polyaminoamide of between 0.5:1 and 1.8:1. Such polymers are described in particular in U.S. Pat. Nos. 3,227,615 and 2,961,347.

Polymers of this type are sold in particular under the name Hercosett 57 by the company Hercules Inc. or alternatively under the name PD 170 or Delsette 101 by the company Hercules in the case of the adipic acid/epoxypropyl/diethylenetriamine copolymer.

(7) Cyclopolymers of alkyldiallylamine or of dialkyldiallylammonium, such as the homopolymers or copolymers containing, as main constituent of the chain, units corresponding to formula (I) or (II):

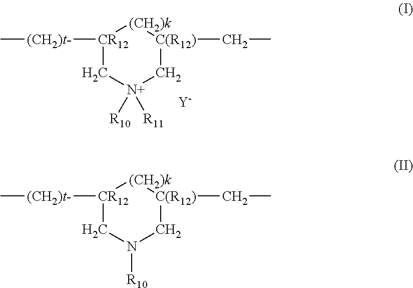

in which formulae k and t are equal to 0 or 1, the sum k+t being equal to 1; R₁₂ denotes a hydrogen atom or a methyl group; R₁₀ and R₁₁, independently of one another, denote an alkyl group having from 1 to 6 carbon atoms, a hydroxyalkyl group in which the alkyl group has preferably 1 to 5 carbon atoms, a lower (C₁-C₄) amidoalkyl group, or else R₁₀ and R₁₁ may, together with the nitrogen atom to which they are attached, denote heterocyclic groups, such as piperidinyl or morpholinyl; Y⁻ is an anion such as bromide, chloride, acetate, borate, citrate, tartrate, bisulfate, bisulfite, sulfate or phosphate. These polymers are especially described in French patent 2 080 759 and in its Certificate of Addition 2 190 406.

R₁₀ and R₁₁, independently of one another, denote preferably an alkyl group having from 1 to 4 carbon atoms.

Among the polymers defined above, mention may be made more particularly of the dimethyldiallylammonium salt (for example chloride) homopolymer sold under the name Merquat 100 by the company Nalco (and homologues thereof of low weight-average molar masses) and the copolymers of diallyldimethylammonium salts (for example chloride) and of acrylamide, sold especially under the names Merquat 550 and Merquat 7SPR.

(8) the quaternary diammonium polymers comprising repeating units corresponding to the formula:

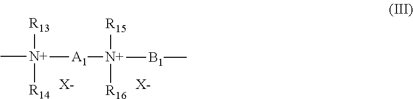

in which formula (III):

R₁₃, R₁₄, R₁₅ and R₁₆, which may be identical or different, represent aliphatic, alicyclic or arylaliphatic radicals comprising from 1 to 20 carbon atoms or lower hydroxyalkylaliphatic radicals, or alternatively R₁₃, R₁₄, R₁₅ and R₁₆, together or separately, constitute, with the nitrogen atoms to which they are attached, heterocycles optionally comprising a second heteroatom other than nitrogen, or alternatively R₁₃, R₁₄, R₁₅ and R₁₆ represent a linear or branched C₁-C₆ alkyl radical substituted with a nitrile, ester, acyl or amide group or a group —CO—O—R₁₇-D or —CO—NH—R₁₇-D where R₁₇ is an alkylene and D is a quaternary ammonium group,

A₁ and B₁ represent polymethylene groups comprising from 2 to 20 carbon atoms, which may be linear or branched, saturated or unsaturated, and which may contain, linked to or intercalated in the main chain, one or more aromatic rings or one or more oxygen or sulfur atoms or sulfoxide, sulfone, disulfide, amino, alkylamino, hydroxyl, quaternary ammonium, ureido, amide or ester groups, and

X⁻ denotes an anion derived from a mineral or organic acid;

A₁, R₁₃ and R₁₅ may form, with the two nitrogen atoms to which they are attached, a piperazine ring; moreover, if A₁ denotes a saturated or unsaturated, linear or branched alkylene or hydroxyalkylene radical, B₁ may also denote a group (CH₂)ₙ—CO-D-OC—(CH₂)ₙ—

in which D denotes:

a) a glycol residue of formula: —O—Z—O—, where Z denotes a linear or branched hydrocarbon-based radical or a group corresponding to one of the following formulae:

—(CH₂—CH₂—O)ₓ—CH₂—CH₂—

—[CH₂—CH(CH₃)—O]_{y}—CH₂—CH(CH₃)—

where x and y denote an integer from 1 to 4, representing a defined and unique degree of polymerization or any number from 1 to 4 representing an average degree of polymerization;

b) a bis-secondary diamine residue such as a piperazine derivative;

c) a bis-primary diamine residue of formula: —NH—Y—NH—, where Y denotes a linear or branched hydrocarbon-based radical, or alternatively the divalent radical

—CH₂—CH₂—S—S—CH₂—CH₂—;

d) a ureylene group of formula: —NH—CO—NH—.

Preferably, X⁻ is an anion such as chloride or bromide.

These polymers have a number-average molecular mass of between 1000 and 100 000.

Polymers of this type are described in particular in French patents 2 320 330, 2 270 846, 2 316 271, 2 336 434 and 2 413 907 and U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945 and 4,027,020.

Use may be made more particularly of polymers that are composed of repeating units corresponding to the formula:

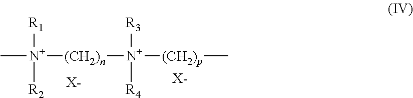

in which R₁, R₂, R₃ and R₄, which may be identical or different, denote an alkyl or hydroxyalkyl radical containing from 1 to 4 carbon atoms approximately, n and p are integers ranging from 2 to 20 approximately, and X⁻ is an anion derived from a mineral or organic acid.

One particularly preferred compound of formula (IV) is that for which R₁, R₂, R₃ and R₄ represent a methyl radical and n=3, p=6 and X═Cl, which is called Hexadimethrine chloride according to INCI nomenclature (CTFA).

(9) Polyquaternary ammonium polymers comprising units of formula (V):

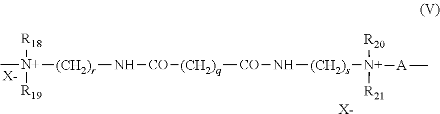

in which formula:

R₁₈, R₁₉, R₂₀ and R₂₁, which may be identical or different, represent a hydrogen atom or a methyl, ethyl, propyl, β-hydroxyethyl, β-hydroxypropyl or —CH₂CH₂(OCH₂CH₂)ₚOH group,

where p is equal to 0 or to an integer between 1 and 6, with the proviso that R₁₈, R₁₉, R₂₀ and R₂₁ do not simultaneously represent a hydrogen atom,

r and s, which may be identical or different, are integers between 1 and 6,

q is equal to 0 or to an integer between 1 and 34,

X⁻ denotes an anion such as a halide,

A denotes a dihalide radical or preferably represents —CH₂—CH₂-O—CH₂—CH₂—.

Such compounds are described especially in patent application EP-A-122 324.

Among these, mention may be made, for example, of the products Mirapol® A 15, Mirapol® AD1, Mirapol® AZ1 and Mirapol® 175, sold by the company Miranol.

(10) Quaternary polymers of vinylpyrrolidone and of vinylimidazole, for instance the products sold under the names Luviquat® FC 905, FC 550 and FC 370 by the company BASF.

(11) Polyamines such as Polyquart® H sold by Cognis, referred to under the name Polyethylene glycol (15) tallow polyamine in the CTFA dictionary.

Other cationic polymers that may be used in the context of the invention are cationic proteins or cationic protein hydrolysates, polyalkyleneimines, in particular polyethyleneimines, polymers comprising vinylpyridine or vinylpyridinium units, condensates of polyamines and of epichlorohydrin, quaternary polyureylenes and chitin derivatives.

Among the cationic polymers mentioned above, which are suitable in the invention, the ones that may preferably be used are quaternary cellulose ether derivatives such as the products sold under the name “JR 400” by the company Amerchol, cationic cyclopolymers, in particular dimethyldiallylammonium salt (for example chloride) homopolymers or copolymers, products sold under the names Merquat 100, Merquat 550 and Merquat S by the company Nalco, and homologues thereof of low weight-average molecular weights, quaternary polymers of vinylpyrrolidone and of vinylimidazole, optionally crosslinked homopolymers or copolymers of methacryloyloxy(C₁-C₄)alkyltri(C₁-C₄)alkylammonium salts, and mixtures thereof.

The cationic polymer(s) are generally present in concentrations ranging from 0.01% to 20% by weight, preferably from 0.05% to 10% by weight and more particularly from 0.1% to 5% by weight, relative to the total weight of the composition.

The composition according to the invention may also especially comprise at least one silicone. This silicone may be linear, branched or cyclic, volatile or non-volatile, and organomodified or non-organomodified.

The term “cosmetically acceptable medium” means a medium that is compatible with keratin materials, especially keratin fibres such as the hair, but which also has a pleasant odour, appearance and feel. The medium is preferably aqueous.

The cosmetically acceptable medium is formed from water or from a mixture of water and at least one organic solvent chosen from C₁-C₄ lower alcohols, such as ethanol, isopropanol, tert-butanol or n-butanol; polyols such as glycerol, propylene glycol and polyethylene glycols.

Preferably, the composition comprises from 70% to 95% by weight of water relative to the total weight of the composition.

The pH of the compositions according to the invention is generally between 2 and 11, preferably between 3 and 10 and better still between 4 and 8.

The composition according to the invention may also comprise additives chosen from anionic polymers, nonionic polymers, amphoteric polymers, non-associative polymeric thickeners, non-polymeric thickeners, nacreous agents, opacifiers, dyes or pigments, fragrances, mineral, plant or synthetic oils, waxes, vitamins, UV-screening agents, free-radical scavengers, antidandruff agents, preserving agents, pH stabilizers and solvents, and mixtures thereof.

A person skilled in the art will take care to select the optional additives and the amount thereof such that they do not harm the properties of the compositions of the present invention.

These additives are generally present in the composition according to the invention in an amount ranging from 0 to 20% by weight relative to the total weight of the composition.

The compositions may be packaged in various forms, especially in bottles, in pump bottles or in aerosol containers so as to apply the composition in vaporized form or in the form of a mousse. The compositions may also impregnate applicators, especially gloves or wipes.

The present invention also relates to a process for washing keratin materials, which consists in applying an effective amount of a composition as defined above to the keratin materials, and in rinsing, for example with water, after an optional leave-on time.

The examples that follow are given as illustrations of the present invention. Unless otherwise mentioned, all the amounts indicated are expressed as weight percentages.

### EXAMPLE 1

The following shampoo composition was prepared:

| Composition | Invention | |
|---|---|---|
| Sodium lauryl ether sulfate (70/30 C₁₂/C₁₄) | 4.9 | g AM |
| containing 2.2 mol of ethylene oxide | | |
| (Texapon AOS 225 UP from Cognis) | | |
| Sodium lauryl sulfate (70/30 C₁₂/C₁₄) | 2.6 | g AM |
| (Texapon LS 35 from Cognis) | | |
| Cocamidopropylbetaine (Dehyton PK 45 | 3.05 | g AM |
| from Cognis) | | |
| 2-Octyldodecanol | 2 | g |
| PEG-136/Steareth-100/HDI Copolymer | 2 | g |
| (Rheolate FX 1100 from Elementis) | | |
| Polyquaternium-10 (Polymer JR400 LT | 0.4 | g |
| from Amerchol) | | |
| Glycerol | 0.5 | g |
| Preserving agents, fragrance | q.s. | |
| pH agent q.s. | pH 5.3 | |
| Demineralized water q.s. | 100 | g |

* as Active material (AM)

The composition in accordance with the invention of Example 1 is thickened and has a smooth, homogeneous appearance, unlike a comparative composition not comprising Rheolate FX 1100, which is fluid.
The composition was applied to premoistened bleached hair (1 g per lock of 2.7 g of hair). This shampoo was worked into a lather and left on the hair for 5 minutes, and then rinsed out with water.

The wet and dried hair, treated with the composition of Example 1, is smooth and easy to disentangle.

### EXAMPLE 2

The following shampoo composition is prepared:

| Composition | 2 | |
|---|---|---|
| Sodium lauryl ether sulfate (70/30 C₁₂/C₁₄) | 4.9 | g AM |
| containing 2.2 mol of ethylene oxide | | |
| (Texapon AOS 225 UP from Cognis) | | |
| Sodium lauryl sulfate (70/30 C₁₂/C₁₄) | 2.6 | g AM |
| (Texapon LS 35 from Cognis) | | |
| Cocamidopropylbetaine (Dehyton PK 45 | 3.05 | g AM |
| from Cognis) | | |
| Oleyl alcohol | 1.5 | g |
| PEG-136/Steareth-100/HDI Copolymer | 2 | g |
| (Rheolate FX 1100 from Elementis) | | |
| Polyquaternium-10 (Polymer JR400 LT | 0.4 | g |
| from Amerchol) | | |
| Glycerol | 0.5 | g |
| Preserving agents, fragrance | q.s. | |
| pH agent q.s. | pH 5.3 | |
| Demineralized water q.s. | 100 | g |

* as Active material (AM)

### EXAMPLE 3

The following shampoo composition is prepared:

| Composition | 2 | |
|---|---|---|
| Sodium lauryl ether sulfate (70/30 C₁₂/C₁₄) | 4.9 | g AM |
| containing 2.2 mol of ethylene oxide | | |
| (Texapon AOS 225 UP from Cognis) | | |
| Sodium lauryl sulfate (70/30 C₁₂/C₁₄) | 2.6 | g AM |
| (Texapon LS 35 from Cognis) | | |
| Cocamidopropylbetaine (Dehyton PK 45 | 3.05 | g AM |
| from Cognis) | | |
| 2-Decyl-1-tetradecanol | 1.5 | g |
| PEG-136/Steareth-100/HDI Copolymer | 2 | g |
| (Rheolate FX 1100 from Elementis) | | |
| Polyquaternium-10 (Polymer JR400 LT | 0.4 | g |
| from Amerchol) | | |
| Glycerol | 0.5 | g |
| Preserving agents, fragrance | q.s. | |
| pH agent q.s. | pH 5.3 | |
| Demineralized water q.s. | 100 | g |

* as Active material (AM)

### EXAMPLES 4 AND 5

The following shampoo composition was prepared:

| Composition | 4 | | 5 | |
|---|---|---|---|---|
| Sodium lauryl ether sulfate (70/30 C₁₂/C₁₄) | 4.9 | g AM | 4.9 | g AM |
| containing 2.2 mol of ethylene oxide | | | | |
| (Texapon AOS 225 UP from Cognis) | | | | |
| Sodium lauryl sulfate (70/30 C₁₂/C₁₄) | 4 | g AM | 4 | g AM |
| (Texapon LS 35 from Cognis) | | | | |
| Disodium cocoamphodiacetate (Miranol | 1.3 | g AM | 1.3 | g AM |
| C2M Conc. MP from Rhodia) | | | | |
| 2-Octyldodecanol | 1.5 | g | | |
| Decyltetradecanol | | | 1 | g |
| PEG-136/Steareth-100/HDI Copolymer | 2 | g | 2 | g |
| (Rheolate FX 1100 from Elementis) | | | | |
| Polyquaternium-10 (Polymer JR400 LT | 0.4 | g | 0.4 | g |
| from Amerchol) | | | | |
| Preserving agents, fragrance | q.s. | | q.s. | |
| pH agent q.s. | pH 5.3 | | pH 5.3 | |
| Demineralized water q.s. | 100 | g | 100 | g |

* as Active material (AM)

The compositions in accordance with the invention of Examples 4 and 5 are thickened and have a smooth, homogeneous appearance, unlike a comparative composition not comprising Rheolate FX 1100, which is fluid.

The composition was applied to premoistened bleached hair (1 g per lock of 2.7 g of hair). This shampoo was worked into a lather and left on the hair for 5 minutes, and then rinsed out with water.

The wet and dried hair, treated with the compositions of Examples 4 and 5, is smooth and easy to disentangle.

### EXAMPLES 6 AND 7

The following shampoo compositions are prepared:

| | 6 | | 7 | |
|---|---|---|---|---|
| Sodium lauryl ether sulfate (70/30 C₁₂/C₁₄) | 4.9 | g AM | 4.9 | g AM |
| containing 2.2 mol of ethylene oxide | | | | |
| (Texapon AOS 225 UP from Cognis) | | | | |
| Sodium lauryl sulfate (70/30 C₁₂/C₁₄) | 4 | g AM | 4 | g AM |
| (Texapon LS 35 from Cognis) | | | | |
| Disodium cocoamphodiacetate (Miranol | 1.3 | g AM | 1.3 | g AM |
| C2M Conc. MP from Rhodia) | | | | |
| Cationic guar gum (Jaguar C162 from | 0.15 | g | | |
| Rhodia) | | | | |
| 2-Decyltetradecanol | 0.05 | g | 0.05 | g |
| PEG-136/Steareth-100/HDI Copolymer | 2 | g | 2 | g |
| (Rheolate FX 1100 from Elementis) | | | | |
| Polyquaternium-10 (Ucare Polymer JR400 | | | 0.1 | g |
| LT from Amerchol) | | | | |
| Preserving agents, fragrance | q.s. | | q.s. | |
| pH agent q.s. | pH 5.3 | | pH 5.3 | |
| Demineralized water q.s. | 100 | g | 100 | g |

* as Active material (AM)

16. A composition for washing keratin materials comprising, in a cosmetically acceptable medium:
at least one anionic surfactant, present in an amount ranging from about 3% to about 50% by weight, relative to the total weight of the composition;
at least one liquid C₁₀-C₃₀ alcohol chosen from branched and/or unsaturated liquid C₁₀-C₃₀ alcohols; and
at least one nonionic associative polymer.

17. The composition according to claim 16, wherein the at least one anionic surfactant is chosen from alkyl sulfates, alkyl ether sulphates, alkyl ether carboxylates, and mixtures thereof.

18. The composition according to claim 16, wherein the at least one liquid C₁₀-C₃₀ alcohol comprises from 12 to 30 carbon atoms.

19. The composition according to claim 18, wherein the at least one liquid C₁₀-C₃₀ alcohol has a structure R—OH, in which R denotes a C₁₂-C₂₄ branched alkyl or C₁₂-C₂₄ alkenyl group.

20. The composition according to claim 19, wherein the at least one liquid C₁₀-C₃₀ alcohol is chosen from oleyl alcohol, isocetyl alcohol, isostearyl alcohol, 2-octyl-1-dodecanol, 2-butyloctanol, 2-hexyl-1-dodecanol, 2-decyl-1-tetradecanol, 2-tetradecyl-1-cetanol, and mixtures thereof.

21. The composition according to claim 16, wherein the at least one liquid C₁₀-C₃₀ alcohol is present in an amount ranging from about 0.01% to about 10% by weight, relative to the total weight of the composition.

22. The composition according to claim 16, wherein the at least one nonionic associative polymer is chosen from:
celluloses modified with groups comprising at least one fatty chain comprising at least 8 carbon atoms;
hydroxypropyl guars modified with groups comprising at least one fatty chain comprising at least 8 carbon atoms;
copolymers of vinylpyrrolidone and of fatty-chain hydrophobic monomers comprising at least 8 carbon atoms;
copolymers of C₁-C₆ alkyl methacrylates or acrylates and of amphiphilic monomers comprising at least one fatty chain comprising at least 8 carbon atoms;
copolymers of hydrophilic methacrylates or acrylates and of hydrophobic monomers comprising at least one fatty chain comprising at least 8 carbon atoms;
polyurethane polyethers comprising in their chain both hydrophilic blocks of polyoxyethylenated nature and hydrophobic blocks, which may optionally be aliphatic sequences alone and/or cycloaliphatic and/or aromatic sequences;
polymers with an aminoplast ether backbone bearing at least one fatty chain comprising at least 8 carbon atoms; and
mixtures thereof.

23. The composition according to claim 16, wherein the at least one nonionic associative polymer is chosen from polyurethane polyethers that may be obtained by polycondensation of at least three compounds comprising:
(i) at least one polyethylene glycol comprising from 100 to 180 mol of ethylene oxide and in particular from 120 to 160 mol of ethylene oxide,
(ii) a C₁₆₋₂₀ fatty alcohol polyoxyethylenated with 80 to 120 and in particular 90 to 110 mol of ethylene oxide and especially a polyoxyethylenated stearyl alcohol comprising from 80 to 120 and in particular 90 to 110 mol of ethylene oxide, and
(iii) an aliphatic diisocyanate.

24. The composition according to claim 16, wherein the at least one nonionic associative polymer is present in an amount ranging from about 0.1% to about 5% by weight, relative to the total weight of the composition.

25. The composition according to claim 24, wherein the at least one nonionic associative polymer is present in an amount ranging from about 1.5% to about 4% by weight, relative to the total weight of the composition.

26. The composition according to claim 16, further comprising at least one nonionic surfactant and/or at least one amphoteric surfactant.

27. The composition according to claim 16, wherein the cosmetically acceptable medium is chosen from water, or of a mixture of water and at least one cosmetically acceptable organic solvent.

28. The composition according to claim 16, wherein the composition further comprises at least one cationic polymer.

29. The composition according to claim 16, wherein the composition further comprises at least one silicone.

30. A process for washing, conditioning, or both washing and conditioning, keratinous fibers, said process comprising a step of applying to said keratinous fibers a composition comprising, in a cosmetically acceptable medium:
at least one anionic surfactant, present in an amount ranging from about 3% to about 50% by weight, relative to the total weight of the composition;
at least one liquid C₁₀-C₃₀ alcohol chosen from branched and/or unsaturated liquid C₁₀-C₃₀ alcohols; and
at least one nonionic associative polymer.

31. The process according to claim 30, further comprising a step of rinsing the keratinous fibers.
